(19) United States
(12) Patent Application Publication
(10) Pub. No.: US 2018/0338846 A1
(43) Pub. Date: Nov. 29, 2018

(54) DEVICES AND METHODS FOR MULTI-LUMEN ACCESS AND DRAINAGE

(71) Applicant: Boston Scientific Scimed, Inc., Maple Grove, MN (US)

(72) Inventors: Martyn G. Folan, Galway (IE); Michael Walsh, Galway (IE); Javier Palomar-Moreno, Galway (IE); Emma-Jane Mooney, Galway (IE); Cormac Finnegan, Galway (IE); Michael G. Walsh, Galway (IE); Thomas M. Keating, Galway (IE)

(21) Appl. No.: 15/988,547

(22) Filed: May 24, 2018

Related U.S. Application Data
(60) Provisional application No. 62/511,171, filed on May 25, 2017.

(51) Int. Cl. A61F 2/856; A61M 27/00; A61M 39/22; A61B 17/34; A61F 2/966

(52) U.S. Cl. CPC A61F 2/856; A61M 27/002; A61M 39/22; A61B 2017/00278; A61F 2/966; A61B 2017/3425; A61B 17/3423

(57) ABSTRACT

The present disclosure relates generally to the field of medical devices and establishing fluid communication between body lumens. In particular, the present disclosure relates to minimally invasive endoscopy devices and methods for body lumen access and/or drainage, and devices and methods for creating an open flow passage between two or more body lumens.

### CROSS REFERENCE TO RELATED APPLICATIONS

This application claims the benefit of priority under 35 U.S.C. § 119 to U.S. Provisional Patent Application Ser. No. 62/511,171, filed on May 25, 2017, which is incorporated by reference in its entirety for all purposes.

### FIELD

The present disclosure relates generally to the field of medical devices and establishing fluid communication between body lumens. In particular, the present disclosure relates to devices and methods for establishing multi-lumen access and/or drainage between two or more body lumens.

### BACKGROUND

Conventional anastomotic devices are indicated for drainage between apposed and/or adherent tissue walls, including, e.g., transgastric or transduodenal drainage of pancreatic pseudocysts adherent to the gastric or bowel wall, respectively. Although generally effective for their designated purpose(s), these devices tend to establish competing bi-directional fluid flow between body lumens and/or limit the number of medical tools which may be introduced into the distal body lumen. These features are less than ideal for certain transluminal procedures, including, e.g., establishing flow between non-adherent and/or non-adjacent body lumens, draining multiple sites (e.g., cysts) within a body lumen and/or establishing flow between two or more body lumens.

A variety of advantageous medical outcomes may therefore be realized by the devices and/or methods of the present disclosure, which allow bi-directional flow and/or access between two or more adherent or non-adherent body lumens.

### SUMMARY

In one aspect, the present disclosure relates to a medical device comprising an elongate tubular member having a first collapsed configuration, and a second expanded configuration in which a first portion of the elongate tubular member expands into a first retention member defining a first opening, a second portion of the elongate tubular member expands into a second retention member defining a second opening, and a cylindrical saddle region having a circumference and longitudinal axis extending between the first and second retention members. The cylindrical saddle region may include a third opening defined therein. The third opening may be positioned along an outer radius of the cylindrical saddle region when the elongate tubular member moves from a linear configuration to a curved configuration. The third opening of the cylindrical saddle region may be configured to receive a portion of a second medical device. The first retention member, second retention member and cylindrical saddle region may be covered apart from the first, second and third openings. The proximal retention member, distal retention member and cylindrical saddle region may define an open interior passage therethrough. The first retention member may include a first flared flange structure and the second retention member may include a second flared flange structure. The first retention member may include a first single-wall flange structure and the second retention member may include a second single-wall flange structure. The first and second single-wall flange structures may extend perpendicular to the longitudinal axis from the circumference of the cylindrical saddle region. The first retention member may include a first double-wall flange structure and the second retention member may include a second double-wall flange structure. The first and second double-wall flange structures may extend perpendicular to the longitudinal axis from the circumference of the cylindrical saddle region. A diameter of the first and second retention members may be greater than a diameter of the cylindrical saddle region. For example, a diameter of the first and second retention members may be 75-100% greater than a diameter of the cylindrical saddle region. The cylindrical saddle region may include a constant or varying outer diameter. A diameter of the first retention member may be equal to a diameter of the second retention member. A diameter of the first retention member may be less than a diameter of the second retention member. At least one valve may be disposed within the open interior passage and/or the at least one first, second and third openings of the elongate tubular member. The third opening may be positioned at an approximate midpoint of the circumference of the cylindrical saddle region. The cylindrical saddle region may include fourth and fifth openings positioned at different radial locations relative to the longitudinal axis of the cylindrical saddle region. At least a portion of the first retention member may be configured to contact an inner surface of a first body lumen at a first location, at least a portion of the second retention member may be configured to contact the inner surface of the first body lumen at a second location and the third opening of the cylindrical saddle region may be configured to open into a second body lumen. At least a portion of the first retention member may be configured to contact an inner surface of a second distal body lumen at a first location, at least a portion of the second retention member may be configured to contact the inner surface of the second distal body lumen at a second location and the third opening of the cylindrical saddle region may be configured to open into a first proximal body lumen. The first retention member may be configured to contact an inner circumference of a first body lumen, the second retention member may be configured to contact an inner circumference of a second body lumen and the third opening of the cylindrical saddle region may be configured to at least partially overlap a junction of the first and second body lumens with a third body lumen. The first retention member may be configured to contact an inner circumference of a first body lumen, the second retention member may be configured to contact an inner circumference of a second body lumen and the third opening of the cylindrical saddle region may be configured to at least partially overlap a junction of the first and second body lumens with a third body lumen. The third opening of the cylindrical saddle region may be configured to receive a portion of a second medical device disposed within the second body lumen. The third opening of the cylindrical saddle region may be configured to receive a portion of a second medical device within the third body lumen. The first and second body lumens may be portions of the same body lumen, and the third body lumen is a different body lumen. The first and second body lumens may be different branches of the same body lumen, and the third body lumen is a different body lumen.

In one aspect, the present disclosure relates to a method comprising advancing a medical device into a first body lumen, wherein the medical device includes an elongate tubular member having a first collapsed configuration and a second expanded configuration, advancing the medical device into a second body lumen through an opening in the first body lumen at a first location and an opening in the second body lumen at a first location, further advancing the medical device from the second body lumen into the first body lumen through an opening in the second body lumen at a second location and an opening in the first body lumen at a second location, expanding a distal portion of the elongate tubular member to the second configuration such that a distal retention member of the member is deployed within the first body lumen at the second location, and expanding a proximal portion of the elongate tubular member to the second configuration such that a proximal retention member of the member is deployed within the first body lumen at the first location. The elongate tubular member may include a cylindrical saddle region extending between the proximal and distal retention members, with openings at the distal and proximal portions open into the first body lumen, wherein an opening of the cylindrical saddle region opens into the second body lumen when the proximal and distal retention members are disposed within the first body lumen.

In one aspect, the present disclosure relates to a method comprising advancing a medical device into a first body lumen, wherein the medical device includes an elongate tubular member having a first collapsed configuration and a second expanded configuration, advancing the medical device into a second body lumen through an opening in the first body lumen at a first location and an opening in the second body lumen at a first location, further advancing the medical device from the second body lumen into the first body lumen through an opening in the second body lumen at a second location and an opening in the first body lumen at a second location, further advancing the medical device from the first body lumen into the second body lumen through the opening in the first body lumen at the first location and the opening in the second body lumen at the first location, deploying a distal retention member of the medical device within the second body lumen at the first location and deploying a proximal retention member of the medical device within the second body lumen at the second location. The elongate tubular member may include a cylindrical saddle region extending between the proximal and distal retention members, with openings at the distal and proximal portions open into the second body lumen, and wherein an opening of the cylindrical saddle region opens into the first body lumen when the proximal and distal retention members are disposed within the second body lumen.

In one aspect, the present disclosure relates to a method comprising advancing a medical device into a first body lumen, wherein the medical device includes an elongate tubular member having a first collapsed configuration and a second expanded configuration, advancing the medical device from a first body lumen into a second body lumen through an opening in the first body lumen at a first location and an opening in the second body lumen at a first location, further advancing the medical device into a third body lumen through an opening in the second body lumen at a second location and an opening in the third body lumen at a first location, deploying a distal retention member of the medical device within the third body lumen at the first location, and deploying a proximal retention member of the medical device within the first body lumen at the first location. The elongate tubular member may include a cylindrical saddle region extending between the proximal and distal retention members, wherein an opening of the cylindrical saddle region opens into the second body lumen when the distal retention member is disposed within the third body lumen and the proximal retention member is disposed within the first body lumen.

### BRIEF DESCRIPTION OF THE DRAWINGS

Non-limiting embodiments of the present disclosure are described by way of example with reference to the accompanying figures, which are schematic and not intended to be drawn to scale. In the figures, each identical or nearly identical component illustrated is typically represented by a single numeral. For purposes of clarity, not every component is labeled in every figure, nor is every component of each embodiment shown where illustration is not necessary to allow those of ordinary skill in the art to understand the disclosure. In the figures:

FIGS. 1A-1B provide perspective top (FIG. 1A) and side (FIG. 1B) views of a medical device, according to one embodiment of the present disclosure.

FIGS. 2A-2E illustrate exemplary steps for deployment of a medical device between a first and second body lumen, according to one embodiment of the present disclosure.

FIG. 3A provides a perspective view of medical instruments accessing a second body lumen through a medical device, according to one embodiment of the present disclosure.

FIG. 3B provides a perspective view of a valve disposed within an opening of a medical device, according to one embodiment of the present disclosure.

FIGS. 4A-4B provide perspective views of a medical device in a linear configuration (FIG. 4A) and deployed between a first body lumen and second body lumen (FIG. 4B), according to one embodiment of the present disclosure.

FIG. 5 provides a perspective top view of a medical device, according to one embodiment of the present disclosure.

FIG. 6 provides a perspective side view of a medical device, according to one embodiment of the present disclosure.

FIGS. 7A-7B provide perspective views of a medical device in a linear configuration (FIG. 7A) and deployed between a first body lumen and a second body lumen (FIG. 7B), according to one embodiment of the present disclosure.

FIG. 8A illustrates a guidewire path for deploying a medical device between a first body lumen and second body lumen, according to one embodiment of the present disclosure.

FIG. 8B provides a perspective view of a medical device deployed between a first body lumen and a second body lumen, according to one embodiment of the present disclosure.

FIG. 9A illustrates a guidewire path for deploying a medical device between a first body lumen, a second body lumen and a third body lumen, according to one embodiment of the present disclosure.

FIG. 9B provides a perspective view of a medical device deployed between a first body lumen, a second body lumen and a third body lumen, according to one embodiment of the present disclosure.

FIG. 10 provides a perspective view of connected first and second medical devices disposed within respective first and second body lumen portions, according to one embodiment of the present disclosure.

FIG. 11 provides a perspective view of connected first and second medical devices disposed within respective first and second body lumen portions, according to one embodiment of the present disclosure.

### DETAILED DESCRIPTION

The present disclosure is not limited to the particular embodiments described. The terminology used herein is for the purpose of describing particular embodiments only, and is not intended to be limiting beyond the scope of the appended claims. Unless otherwise defined, all technical terms used herein have the same meaning as commonly understood by one of ordinary skill in the art to which the disclosure belongs.

Although embodiments of the present disclosure are described with specific reference to medical devices (e.g., stents, etc.) and methods for drainage of (or access to) a pancreatic pseudocyst, it should be appreciated that such devices and methods may be used in a variety of medical procedures (e.g., external biliary drain conversion, enteroenterostomy, gastrojejumostomy, gastroduodenostomy and gastroileostomy, etc.) to establish and/or maintain a temporary or permanent open flow passage between or drainage from a variety of body organs, lumens, vessels, fistulas and spaces (e.g., the dermis, stomach, duodenum, gallbladder, bladder, kidneys, walled-off pancreatic necrosis (WOPN), abscesses, etc.). Moreover, such medical devices are not limited to drainage, but may facilitate access to organs, vessels or body lumens for other purposes, such as creating a path to divert or bypass fluids or solids from one location to another, removing obstructions and/or delivering therapy, including non-invasive manipulation of the tissue within the organ and/or the introduction of pharmacological agents via the open flow passage.

As used herein, the singular forms “a,” “an,” and “the” are intended to include the plural forms as well, unless the context clearly indicates otherwise. It will be further understood that the terms “comprises” and/or “comprising,” or “includes” and/or “including” when used herein, specify the presence of stated features, regions, steps elements and/or components, but do not preclude the presence or addition of one or more other features, regions, integers, steps, operations, elements, components and/or groups thereof.

As used herein, the term “distal” refers to the end farthest away from the medical professional when introducing a device into a patient, while the term “proximal” refers to the end closest to the medical professional when introducing a device into a patient.

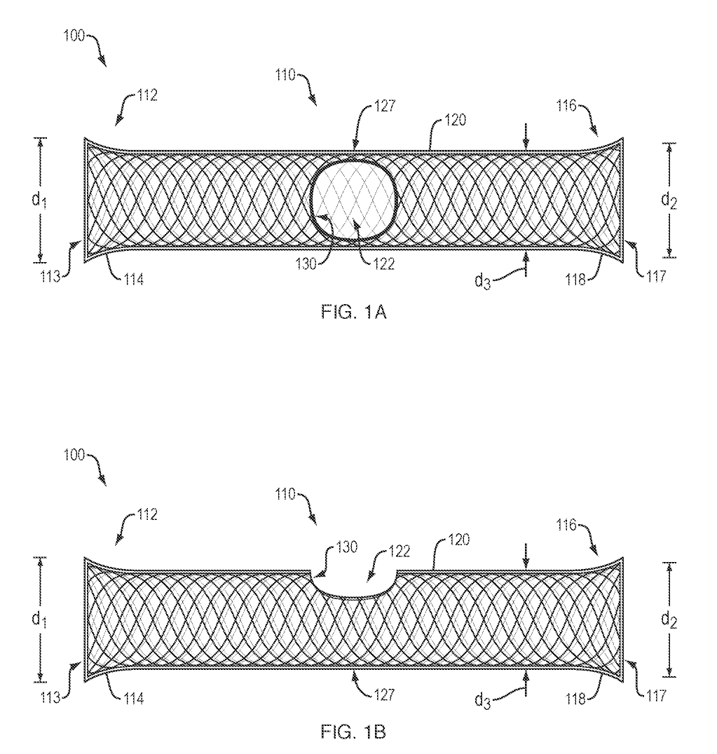

In various embodiments, the present disclosure relates to devices and methods for creating an open flow or access passage between two or more body lumens. Referring to FIGS. 1A-1B, in one embodiment, a medical device 100 of the present disclosure may include an elongate tubular member 110 configured to move between a first (e.g., constrained, collapsed, non-expanded) configuration and a second (e.g., non-constrained, expanded) configuration. In the second configuration, a first (e.g., proximal) portion 112 of the elongate tubular member 110 may form a first (e.g., proximal) retention member 114 defining a first (e.g., proximal) opening 113, and a second (e.g., distal) portion 116 of the elongate tubular member 110 may form a second (e.g., distal) retention member 118 defining a second (e.g., distal) opening 117. A cylindrical saddle region 120 may extend between the first and second retention members 114, 118 to define an open interior passage 130 (e.g., channel, lumen, etc.) therebetween. The cylindrical saddle region 120 may further include a third opening 122 formed therein. For example, a third opening 122 may be formed within the saddle region at an approximate midpoint 127 of the cylindrical saddle region 120 (e.g., halfway between the first and second retention members). In addition, or alternatively, the third opening 122 may be formed within the saddle regions at a proximal or distal portion of the cylindrical saddle region (not shown).

The first and second retention members 114, 118 may form respective first and second flared ends (e.g., flared flange structures) configured to atraumatically engage, e.g., the tissue wall of a first body lumen at separate (e.g., first and second) locations, or the tissue wall of a second body lumen at separate locations, or the tissue wall of first body lumen and third body lumen spanning a second body lumen, as discussed below. In one embodiment, an outer diameter d₁ of the first retention member 114 may be equal to an outer diameter d₂ of the second retention member 118. The cylindrical saddle region 120 may include a constant outer diameter d₃ extending between the first and second retention members 114, 118, wherein the diameter d₃ of the cylindrical saddle region is less than the diameters d₁ and d₂ of the first and second retention members 114, 118. For example, outer diameters d₁ and d₂ may be approximately 7.0 mm to approximately 30 mm, and outer diameter d₃ may be approximately 3.0 mm to approximately 15.0 mm. For example, in one or more embodiments, the first and second retention members may include an outer diameter d₁ and d₂ that is as much as 75%-100% greater than an outer diameter d₃ of the cylindrical saddle region. In one embodiment, the cylindrical saddle region 120 may include a length of approximately 40-50 mm.

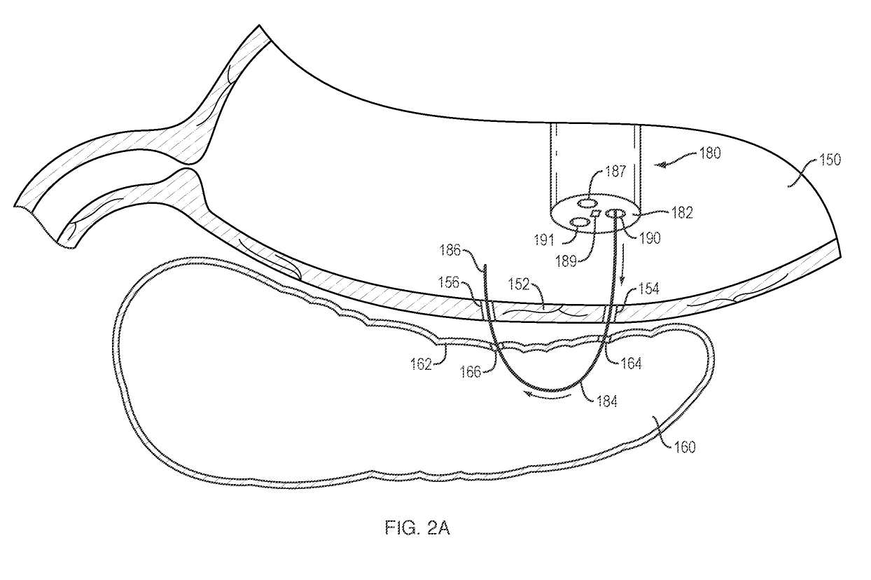

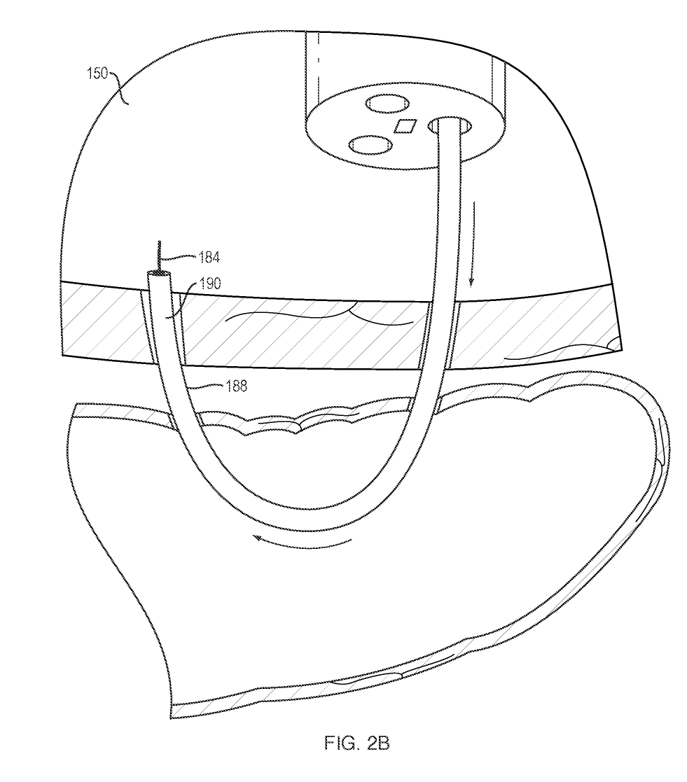

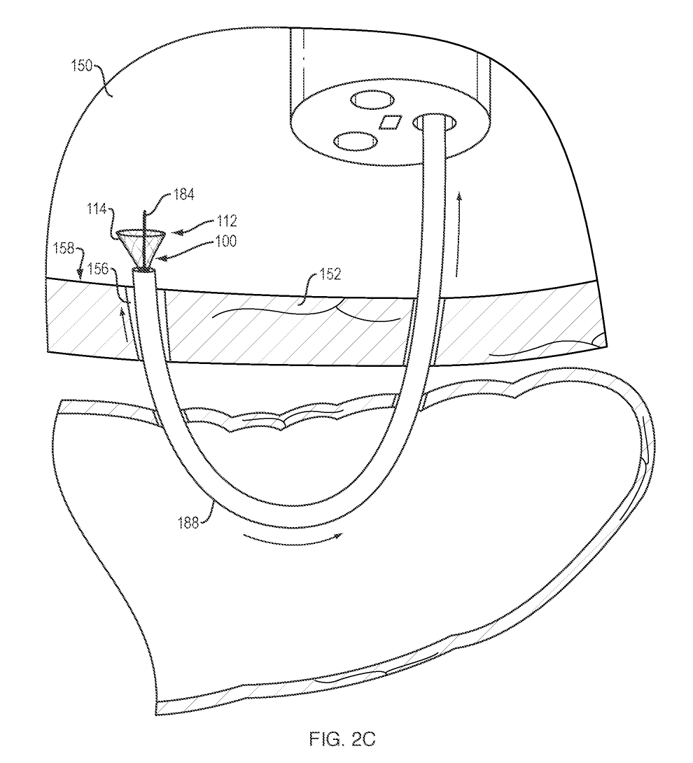

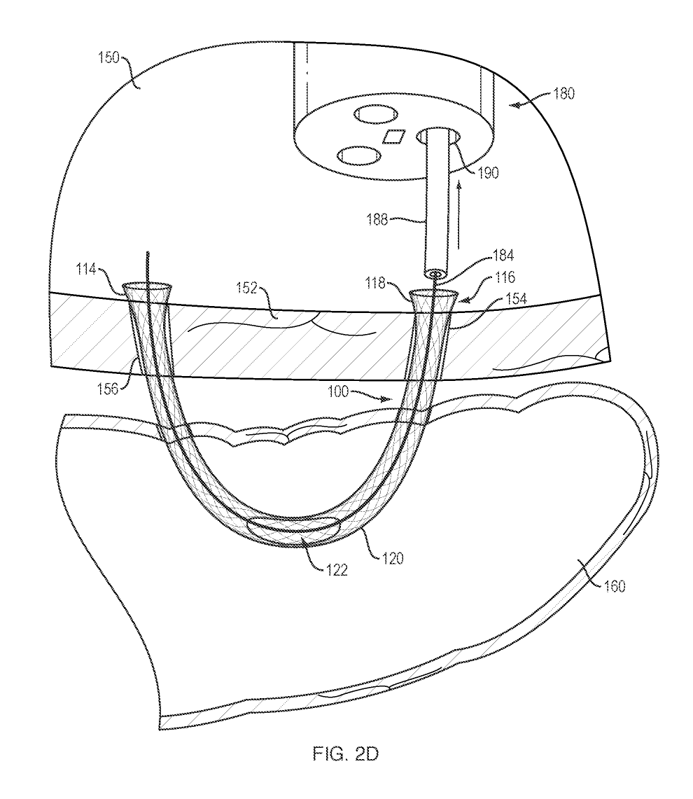

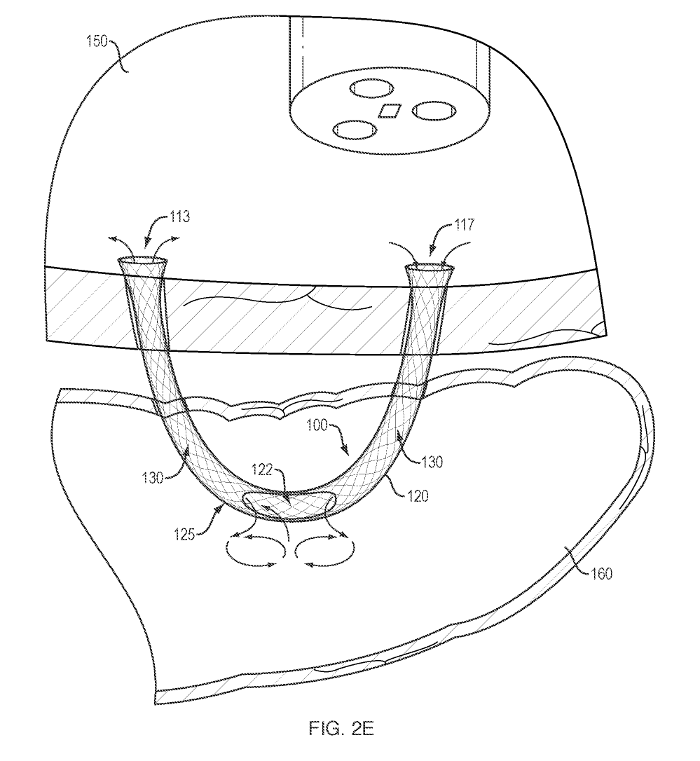

In one embodiment, the first retention member 114 may be configured to contact an inner surface of a first body lumen at a first location, the second retention member 118 may be configured to contact an inner surface of the first body lumen at a second location and the cylindrical saddle region 120 and third opening 122 may be configured to open into a second body lumen. Referring to FIG. 2A, in use and by way of example, an endoscope 180 may be advanced through the esophagus into a first body lumen 150 (e.g., stomach). A distal end 182 of the endoscope 180 may include may include a camera 187, light source 191 and ultrasound transducer 189. Using the endoscopic view (e.g., the light source 191 and camera 187), the distal end 182 of the endoscope 180 may be positioned adjacent to a tissue wall 152 of the first body lumen 150 which is in the vicinity of the tissue wall 162 of a second body lumen 160 (e.g., pancreatic pseudocyst). The second body lumen 160 may then be imaged through the first tissue wall 152 by switching the endoscope 180 from the endoscopic view to an ultrasound view, e.g., turning off the light source 191 and turning on the ultrasound transducer 189. A tissue-penetrating element (e.g., needle, etc.; not shown) comprising a proximal end, a sharpened distal end and a lumen extending therebetween, may then be advanced through a working channel 190 of the endoscope 180 such that the sharpened distal end penetrates the first tissue wall 152 at a first location 154, penetrates the second tissue wall 162 at a first location 164 and extends into the second body lumen 160. The tissue-penetrating element may then be bent (e.g., torqued or retroflexed) and advanced within the second body lumen 160 such that the sharpened distal end penetrates the second tissue wall 162 at a second location 166 (e.g., different than the first location 164), penetrates the first tissue wall 152 at a second location 156 (e.g., different than the first location 154) and extends into the first body lumen 150. In one embodiment, the tissue-penetrating element may include a 19 or 21-gauge needle used for fine-needle aspiration (FNA) or fine-needle biopsy (FNB) procedures, as are known in the art. Alternatively, a distal end of the tissue-penetrating element may include an electrocautery element, as are known in the art. A guidewire 184 may be advanced through the lumen of the tissue-penetrating element to position a distal portion 186 of the guidewire 184 within the first body lumen 150 (FIG. 2A). A sufficient amount of the guidewire 184 may be advanced through the tissue-penetrating element to form a loop (not shown) within the first body lumen to maintain the proper location of the guidewire throughout the medical procedure. Referring to FIG. 2B, the tissue-penetrating element (not shown) may then be retracted over the guidewire 184, and a sheath 188 may be advanced over the guidewire 184 to position a distal end 190 of the sheath 188 within the first body lumen 150. Referring to FIG. 2C, a medical device delivery system (not shown) may then be advanced over the guidewire 184 and through the sheath 188 to position a distal end of the delivery system within the first body lumen 150. The delivery system may include an inner member with a lumen extending therethrough to receive the guidewire 184, and an outer member disposed coaxially about the inner member. A medical device, such as medical device 100 of FIGS. 1A, 1B, may be loaded onto the delivery system in a first configuration between the inner and outer member. Alternatively, a delivery system for a medical device, such as described here, may be advanced to the desired location over a guidewire (e.g., guidewire 184) without the use of a sheath (e.g. sheath 188). A first portion 112 of the medical device 100 may be exposed from within the delivery system to form a first retention member 114 within the first body lumen 150 at the second location 156 of the tissue wall 152. The delivery system and sheath 188 may then be proximally retracted (e.g., pulled) to place the first retention member 114 in contact with an inner surface 158 of the first body lumen 150. Referring to FIG. 2D, the sheath 188 and delivery system may be further proximally retracted through the second body lumen 160 to position their respective distal ends within the first body lumen 150 at the first location 154 of the first tissue wall 152. A second portion 116 of the medical device 100 may be exposed from within the delivery system to form a second retention member 118 within the first body lumen 150 at the first location 154 of the tissue wall 152. With the first and second retention members 114, 118 disposed within their respective locations 156, 154 of the first body lumen 150, the medical device 100 may move to a curved or “U-shaped” configuration such that a portion of the cylindrical saddle region 120, including the third opening 122, is disposed within the second body lumen 160, thereby providing an open flow path or access path between the first and second body lumens 150, 160. With the medical device 100 properly deployed, the delivery system may be removed over the guidewire 184 and through the sheath 188. The sheath 188 and guidewire 184 may then also be retracted through the medical device and into the working channel 190 of the endoscope 180. The endoscope 180, delivery system and sheath 188 may then be removed from the patient. In an alternative embodiment, without sheath 188, a delivery system for a medical device, may be retracted along the guidewire and along the same path, with the first and second retention members deployed, as described above, by retracting the outer member from about the inner member of the delivery system to deploy the medical device.

Referring to FIG. 2E, with the guidewire and sheath removed from within the medical device 100, the first opening 113, second opening 117 and third opening 122 of the cylindrical saddle region 120, may allow non-competing (e.g., bi-directional) flow and/or circulation of fluids between the first and second body lumens 150, 160. FIG. 2E depicts an exemplary flow path that proceeds from the first body lumen 150 into the second body lumen 160 through the second opening 117 and third opening 122 of the cylindrical saddle region 120 (e.g., through a portion of the open interior passage 130), and then from the second body lumen 160 into the first body lumen 150 through the third opening 122 of the cylindrical saddle region through the first opening 113 (e.g., through another portion of the open interior passage 130). In various embodiments, the flow path may include an opposite direction of flow and/or various combinations of flow directions. For example, the direction of flow between the first and second body lumens 150, 160 may vary and/or change depending on the viscosity of the fluid(s) drained from the second body lumen 160. These viscosities may change throughout the course of the drainage procedure, e.g., as high viscosity fluids are removed and/or additional fluids are introduced into (e.g., flushed through) the first and second 150, 160 body lumens. In addition, the direction of flow may vary based on the gravitational forces exerted on or between the body lumens, e.g., as the patient moves, sits, stands or lays down. In one embodiment, when the medical device 100 is in the curved or U-shaped configuration, the third opening 122 may be positioned or oriented within an outer radius 125 of the cylindrical saddle region 120 (e.g., facing away from the first body lumen), thereby maximizing a diameter of the third opening 122. In various embodiments, the third opening 122 may be positioned or oriented within an inner radius of the cylindrical saddle region (e.g., facing toward the first body lumen), or between the inner radius and outer radius of the cylindrical saddle region (not shown).

Although FIGS. 2A-2E outline exemplary steps by which a medical device 100 of the present disclosure may be deployed between first and second body lumens 150, 160, in various embodiments the same (or similar) steps may be used to deploy any of the medical devices 200, 300, 400, as discussed below.

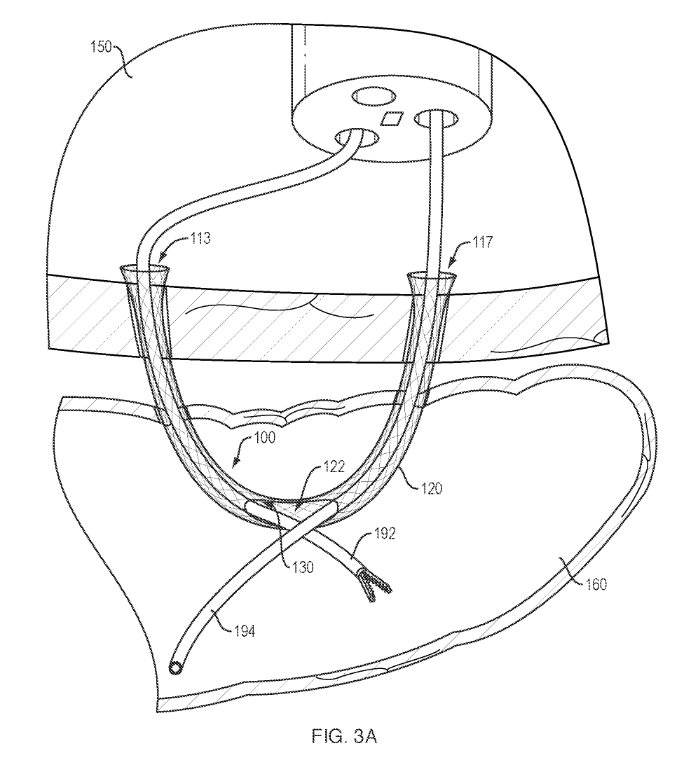

Referring to FIG. 3A, in addition to providing a bi-directional flow path, the open interior passage 130 of the medical device 100 may provide separate access paths between the first and second body lumens 150, 160 for the simultaneous introduction and manipulation of two or more medical tools. For example, a first medical tool 192 (e.g., forceps, cutting element, etc.) may be advanced from the first body lumen 150 into the second body lumen 160 through the first opening 113 and third opening 122 of the cylindrical saddle region 120 (e.g., through a portion of the open interior passage 130), and a second medical tool 194 (e.g., suction tube, lavage tube, etc.) may be advanced from the first body lumen 150 into the second body lumen 160 through the second opening 117 and third opening 122 of the cylindrical saddle region 120 (e.g., through another portion of the open interior passage). In various embodiments, the ability to simultaneously manipulate two or more medical tools within the second body lumen 160 may allow the medical professional to triangulate the position of each medical device within the second body lumen 160 for a safer and more efficient procedure. It should be appreciated that the present disclosure is not limited to the use of first and second medical tools 192, 194 with the medical device 100 depicted in FIGS. 1A-1B, but may be used in a similar fashion with any of the medical devices 200, 300, 400, as discussed below.

In various embodiments, the medical device 100 may include one or more valves (e.g., duck-bill valve, slit valve, etc.) moveable between closed and opened configurations to block or prevent or minimize the flow of fluids unless or until the medical professional determines that the valve should be opened (e.g., by inserting a drainage tube, medical device, etc.) or a certain threshold (e.g., pressure) that built into the valve is reached that allows it to open. Examples of suitable valves are described in U.S. Patent Publication No. 2012/0226243, the contents of which is hereby incorporated by reference in its entirety. Such valves may comprise a variety of suitable biocompatible and non-degradable materials, including any of the polymers discussed herein. Referring to FIG. 3B, in one embodiment, a valve 140 may be positioned within the third opening 122 of the cylindrical saddle region 120. In other embodiments, one or more valves 140 may be positioned at various locations along the open interior passage 130, including, but not limited to, the first and second openings 113, 117. It should be appreciated that the present disclosure is not limited to including one or more valves within the medical device 100 depicted in FIGS. 1A-1B, but may be included or incorporated in a similar fashion within any of the medical devices 200, 300, 400, 500, 600, as discussed below. Valves according to various embodiments may be employed to control the direction of flow in one way or another (e.g., downstream or upstream flow from the valve), or may be employed to control bidirectional flow in both directions.

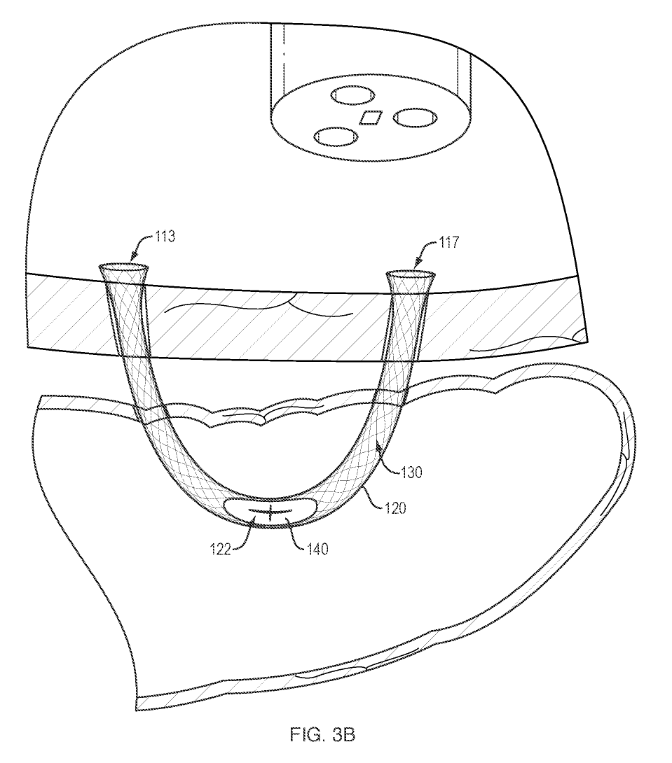

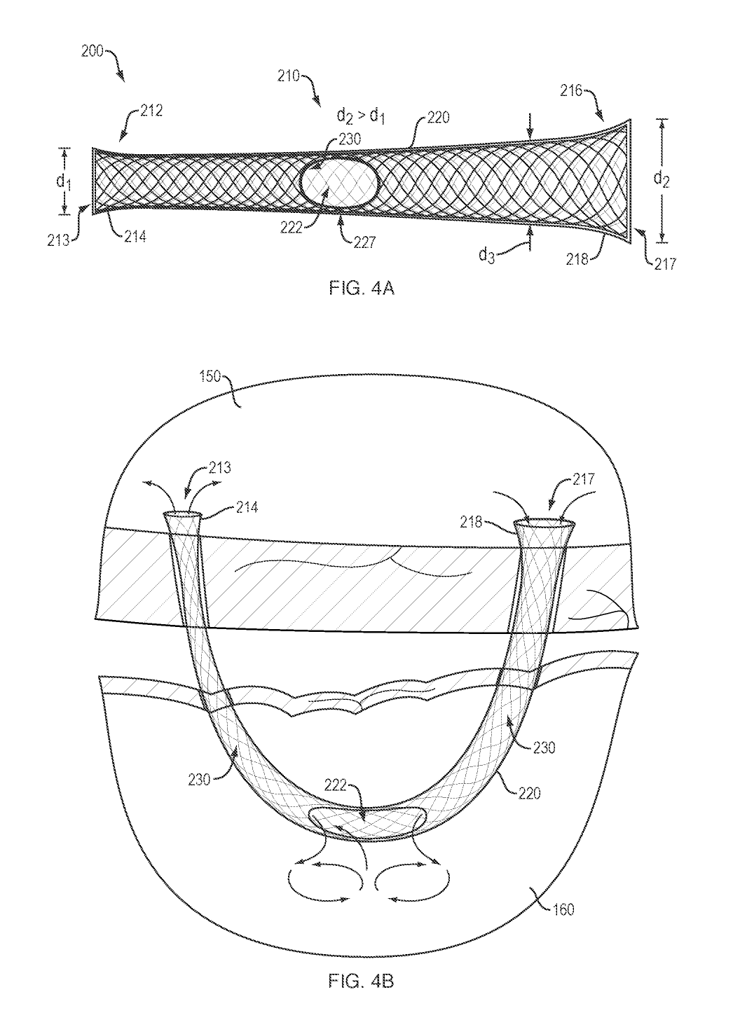

Referring to FIG. 4A, in one embodiment, a medical device 200 of the present disclosure may include an elongate tubular member 210 configured to move between a first (e.g., constrained, collapsed, non-expanded) configuration and a second (e.g., non-constrained, expanded) configuration. In the second configuration, a first (e.g., proximal) portion 212 of the elongate tubular member 210 may form a first (e.g., proximal) retention member 214 defining a first (e.g., proximal) opening 213, and a second (e.g., distal) portion 216 of the elongate tubular member 210 may form a second (e.g., distal) retention member 218 defining a second (e.g., distal) opening 217. The first and second retention members 214, 218 may form respective first and second flared ends (e.g., flared flange structures) configured to atraumatically engage the tissue wall of a first body lumen at separate (e.g., first and second) locations, as discussed above. A cylindrical saddle region 220 of elongate tubular member may extend between the first and second retention members 214, 218 to define an open interior passage 230 (e.g., channel, lumen, etc.) therebetween. The cylindrical saddle region 220 may further include a third opening 222 formed therein. For example, the third opening 222 may be formed within the saddle region at an approximate midpoint 227 of the cylindrical saddle region 220 (e.g., halfway between the first and second retention members). In addition, or alternatively, the third opening 222 may be formed within the saddle region at a proximal or distal portion of the cylindrical saddle region (not shown). The cylindrical saddle region may include an outer diameter d₃ that varies (e.g., tapers) along its length between the first and second retention members 214, 218. An outer diameter d₂ of the second retention member 218 may be greater an outer diameter d₁ of the first retention member 214 and the outer diameter d₃ of the cylindrical saddle region 220.

Referring to FIG. 4B, the first opening 213, second opening 217 and third opening 222 may allow non-competing (e.g., bi-directional) flow and/or circulation of fluids between the first and second body lumens 150, 160. For example, in one embodiment, the larger outer diameter d₂ of the second retention member 218 as compared to the outer diameter d₁ of the first retention member 214 may provide a preferential flow path that proceeds from the first body lumen 150 into the second body lumen 160 through the second opening 217 and third opening 222 of the cylindrical saddle region 220 (e.g., through a portion of the open interior passage 230), and then from the second body lumen 160 into the first body lumen 150 through the third opening 222 of the cylindrical saddle region 220 and first opening 213 (e.g., through another portion of the open interior passage 230). In addition, or alternatively, the larger outer diameter d₂ of the second retention member 218 as compared to the outer diameter d₁ of the first retention member 214 may provide additional anchoring at or between the first and second body lumens 150, 160. The device 200 may be placed between the first body lumen and second body lumen, in a procedure similar to that described above with respect to medical device 100.

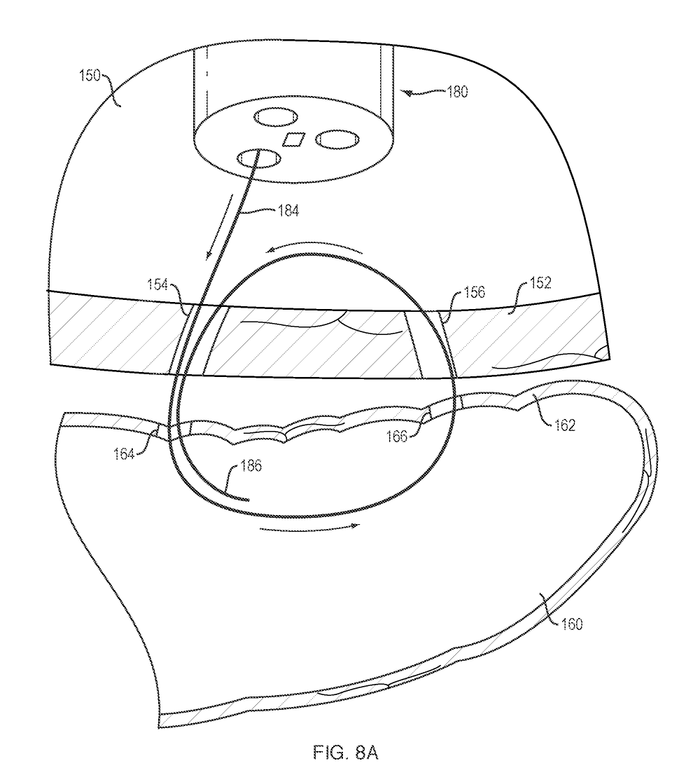

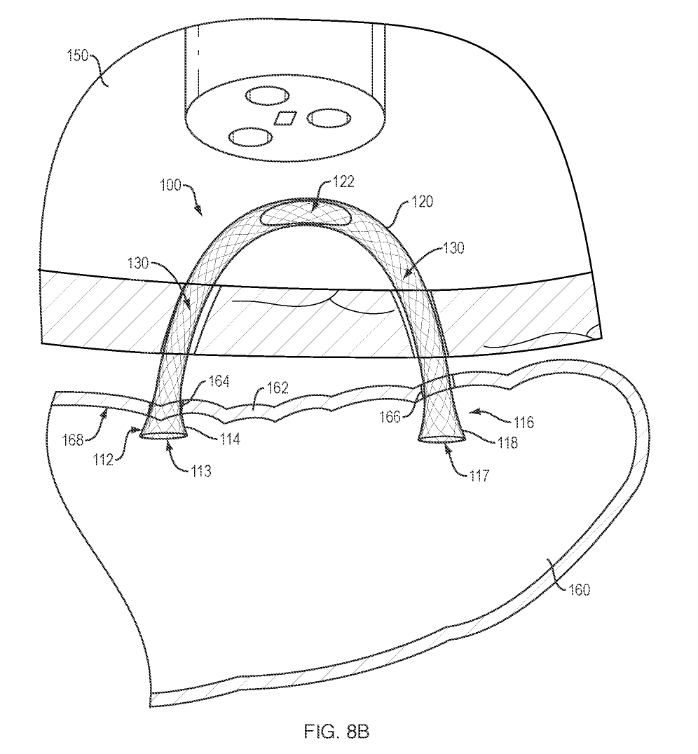

Alternatively, with reference to FIGS. 8A-8B, in one embodiment, the medical device 200 may provide a preferential flow path that proceeds from the second body lumen 160 into the first body lumen 150 through the second opening 217 and third opening 222 of the cylindrical saddle region 220 (e.g., through a portion of the open interior passage 230), and then from the first body lumen 150 into the second body lumen 160 through the third opening 222 of the cylindrical saddle region 220 and first opening 213 (e.g., through another portion of the open interior passage 230. The procedure described below, with reference to FIGS. 8A-8B, may be used to place medical devices 100, 200 in a similar fashion.

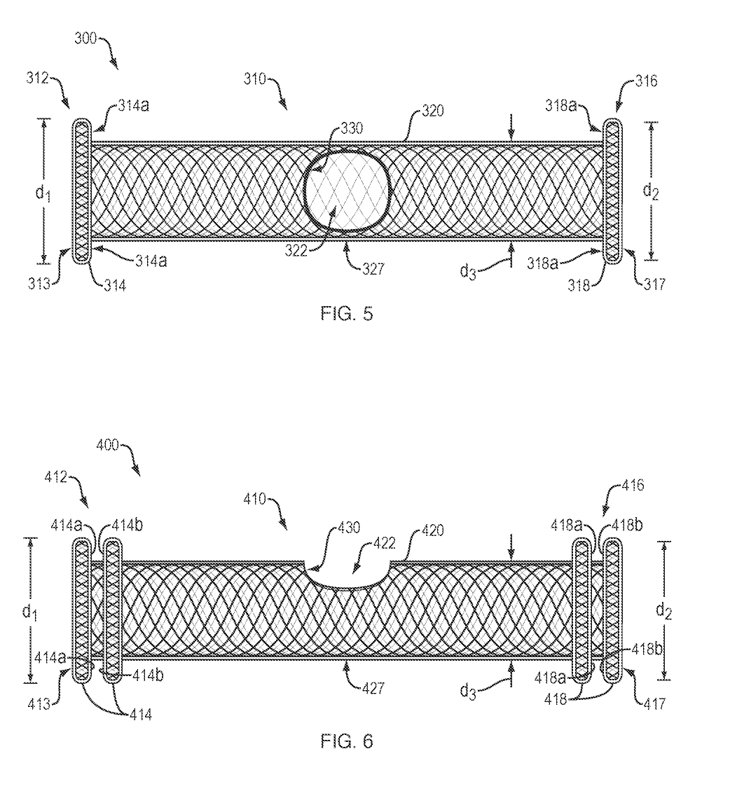

Referring to FIG. 5, in one embodiment, a medical device 300 of the present disclosure may include an elongate tubular member 310 configured to move between a first (e.g., constrained, collapsed, non-expanded) configuration and a second (e.g., non-constrained, expanded) configuration. In the second configuration, a first (e.g., proximal) portion 312 of the elongate tubular member 310 may form a first (e.g., proximal) retention member 314 defining a first (e.g., proximal) opening 313, and a second (e.g., distal) portion 316 of the elongate tubular member 310 may form a second (e.g., distal) retention member 318 defining a second (e.g., distal) opening 317. A cylindrical saddle region 320 may extend between the first and second retention members 314, 318 to define an open interior passage 330 (e.g., channel, lumen, etc.) therebetween. The cylindrical saddle region 320 may further include a third opening 322 formed therein. For example, the third opening 322 may be formed within the saddle region at an approximate midpoint 327 of the cylindrical saddle region 320 (e.g., halfway between the first and second retention members). In addition, or alternatively, the third opening may be formed within the saddle regions at a proximal or distal portion of the cylindrical saddle region (not shown).

The first and second retention members 314, 318 may form respective first and second single-wall flange structures that extend perpendicular to a circumference of the elongate tubular member 310 to define respective planar surfaces 314a, 318a. In one embodiment, an outer diameter d₁ of the first retention member 314 may be equal to an outer diameter d₂ of the second retention member 318. The cylindrical saddle region 320 may include a constant outer diameter d₃ extending between the first and second retention members 314, 318, wherein the diameter d₃ of the cylindrical saddle region is less than the diameters d₁ and d₂ of the first and second retention members. In some cases, diameters d₁ and d₂ may be up to 75%-100% larger in diameter than diameter d₃ of the cylindrical saddle region. In one embodiment, the medical device 300 may be positioned within a patient such that the respective planar surface 314a, 318a of the first and second single-wall flange structures atraumatically contact (e.g., engage) different portions of an inner surface of a first body lumen. For example, the planar surface 314a of the first single-wall flange structure may contact an inner surface of a first body lumen at one location (e.g., a first location), and the planar surface 318a of the second single-wall flange structure may contact an inner surface of the first body lumen at another location (e.g., a second location). With the first and second retention members 314, 318 disposed within their respective locations of the first body lumen, the medical device 300 may move to a curved or “U-shaped” configuration such that a portion of the cylindrical saddle region 320, including the third opening 322, is disposed within the second body lumen, thereby providing a multi-lumen open flow path or access path between the first and second body lumens. The device 300 may be placed between the first body lumen and second body lumen, in a procedure similar to that described above with respect to medical device 100.

Alternatively, with reference to FIGS. 8A-8B, in one embodiment, the medical device 300 may be positioned within a patient such that the respective planar surface 314a, 318a of the first and second single-wall flange structures atraumatically contact (e.g., engage) different portions of an inner surface of a second body lumen. For example, the planar surface 314a of the first single-wall flange structure may contact an inner surface of a second body lumen at one location (e.g., a first location), and the planar surface 318a of the second single-wall flange structure may contact an inner surface of the second body lumen at another location (e.g., a second location). With the first and second retention members 314, 318 disposed within their respective locations of the second body lumen, the medical device 300 may move to a curved or “U-shaped” configuration such that a portion of the cylindrical saddle region 320, including the third opening 322, is disposed within the first body lumen, thereby providing a multi-lumen open flow path or access path between the first and second body lumens. The procedure described below, with reference to FIGS. 8A-8B, may be used to place medical device 300 in a similar fashion.

Referring to FIG. 6, in one embodiment, a medical device 400 of the present disclosure may include an elongate tubular member 410 configured to move between a first (e.g., constrained, collapsed, non-expanded) configuration and a second (e.g., non-constrained, expanded) configuration. In the second configuration, a first (e.g., proximal) portion 412 of the elongate tubular member 410 may form a first (e.g., proximal) retention member 414 defining a first (e.g., proximal) opening 413, and a second (e.g., distal) portion 416 of the elongate tubular member 410 may form a second (e.g., distal) retention member 418 defining a second (e.g., distal) opening 417. A cylindrical saddle region 420 of the elongate tubular member may extend between the first and second retention members 414, 418 to define an open interior passage 430 (e.g., channel, lumen, etc.) therebetween. The cylindrical saddle region 420 may further include a third opening 422 formed therein. For example, the third opening 422 may be formed within the saddle region at an approximate midpoint 427 of the cylindrical saddle region 420 (e.g., halfway between the first and second retention members). In addition, or alternatively, the third opening may be formed within the saddle regions at a proximal or distal portion of the cylindrical saddle region (not shown).

The first and second retention members 414, 418 may form respective first and second double-wall flange structures that extend perpendicular to a circumference of the elongate tubular member 410. The first double-wall flange structure may define opposing planar surfaces 414a and 414b, and the second double-wall flange structure may define opposing planar surface 418a and 418b. In one embodiment, an outer diameter d₁ of the first retention member 414 may be equal to an outer diameter d₂ of the second retention member 418. The cylindrical saddle region 420 may include a constant outer diameter d₃ extending between the first and second retention members 414, 418, wherein the diameter d₃ of the cylindrical saddle region is less than the diameters d₁ and d₂ of the first and second retention members. In some cases, diameters d₁ and d₂ may be up to 75%-100% larger in diameter than diameter d₃ of the cylindrical saddle region.

In one embodiment, the medical device 400 may be positioned within a patient such that the respective opposing planar surface 414a, 414b, 418a, 418b of the first and second double-wall flange structures atraumatically contact (e.g., engage) different portions of a first body lumen. For example, the opposing planar surface 414a, 414b of the first double-wall flange structure may contact opposite sides (e.g., inner surface and outer surface, respectively) of a first body lumen at one location (e.g., a first location), and the opposing planar surface 418a, 418b of the second double-wall flange structure may contact opposite sides (e.g., inner surface and outer surface, respectively) of the first body lumen at another location (e.g., a second location). Alternatively, the opposing planar surface 414a, 414b of the first double-wall flange structure may contact the respective inner surfaces of adjacent body lumens (e.g., inner surface of a first body lumen, and inner surface of a second body lumen) at one location (e.g., a first location) of each body lumen, and the opposing planar surface 418a, 418b of the second double-wall flange structure may contact the respective inner surfaces of the adjacent body lumens at another location (e.g., a second location) of each body lumen. With the first and second retention members 414, 418 disposed within their respective locations of the first body lumen (or first and second body lumens), the medical device 400 may assume a curved or “U-shaped” configuration such that a portion of the cylindrical saddle region 420, including the third opening 422, is disposed within the second body lumen, thereby providing a multi-lumen open flow path or access path between the first and second body lumens. The device 400 may be placed between the first body lumen and second body lumen, in a procedure similar to that described above with respect to medical device 100.

Alternatively, with reference to FIGS. 8A-8B, in one embodiment, the medical device 400 may be positioned within a patient such that the respective opposing planar surface 414a, 414b, 418a, 418b of the first and second double-wall flange structures atraumatically contact (e.g., engage) different portions of a second body lumen. For example, the opposing planar surface 414a, 414b of the first double-wall flange structure may contact opposite sides (e.g., inner surface and outer surface, respectively) of a second body lumen at one location (e.g., a first location), and the opposing planar surface 418a, 418b of the second double-wall flange structure may contact opposite sides (e.g., inner surface and outer surface, respectively) of the second body lumen at another location (e.g., a second location). With the first and second retention members 414, 418 disposed within their respective locations of the second body lumen, the medical device 400 may assume a curved or “U-shaped” configuration such that a portion of the cylindrical saddle region 420, including the third opening 422, is disposed within the first body lumen, thereby providing a multi-lumen open flow path or access path between the first and second body lumens. The procedure described below, with reference to FIGS. 8A-8B, may be used to place medical device 400 in a similar fashion.

Although the medical device 300 depicted in FIG. 5 includes first and second retention members 314, 318 which form respective first and second single-wall flange structures, and the medical device 400 depicted in FIG. 6 includes first and second retention members 414, 418 which form respective first and second double-wall flange structures, in various embodiments, a medical device of the present disclosure may include a first retention member which forms a single-wall flange structure, and a second retention member which forms a double-wall flange structure, or vice versa.

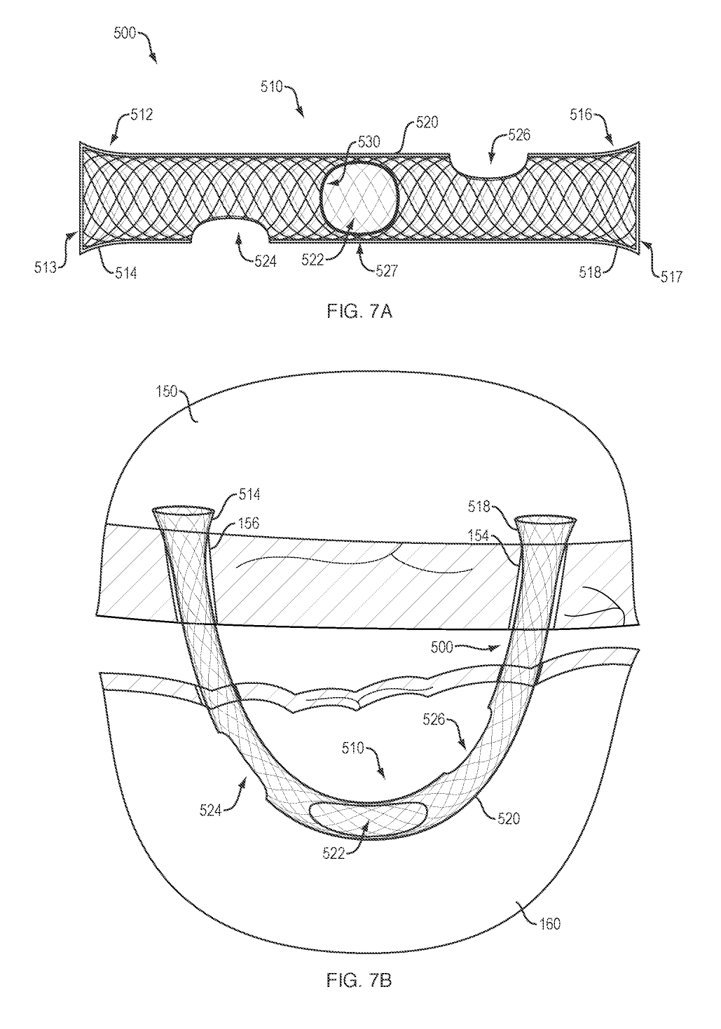

Referring to FIG. 7A, in one embodiment, a medical device 500 of the present disclosure may include an elongate tubular member 510 configured to move between a first (e.g., constrained, collapsed, non-expanded) configuration and a second (e.g., non-constrained, expanded) configuration. In the second configuration, a first (e.g., proximal) portion 512 of the elongate tubular member 510 may form a first (e.g., proximal) retention member 514 defining a first (e.g., proximal) opening 513, and a second (e.g., distal) portion 516 of the elongate tubular member 510 may form a second (e.g., distal) retention member 518 defining a second (e.g., distal) opening 517. A cylindrical saddle region 520 of the elongate tubular member may extend between the first and second retention members 514, 518 to define an open interior passage 530 (e.g., channel, lumen, etc.) therebetween. The cylindrical saddle region 520 may further include a plurality of openings, e.g., third opening 522, fourth opening 524 and fifth opening 526, formed at various locations along a length of the cylindrical saddle region. By way of non-limiting example, the third opening 522 may be formed within the saddle region at an approximate midpoint 527 of the cylindrical saddle region 520 (e.g., halfway between the first and second retention members), the fourth opening 524 may be formed between the third opening 522 and the first retention member 514 and the fifth opening 526 may be formed between the third opening 522 and the second retention member 518. In addition, or alternatively, each of the third, fourth and fifth openings 522, 524, 526 may be positioned at a different radial location about the circumference of the tubular member relative to a longitudinal axis of the cylindrical saddle region 520.

Referring to FIG. 7B, with the first and second retention members 514, 518 disposed within their respective locations 156, 154 of the first body lumen 150, the medical device 500 may assume a curved or “U-shaped” configuration such that a portion of the cylindrical saddle region 520, including the third, fourth and fifth openings 522, 524, 526 are disposed within the second body lumen 160, thereby providing a multi-lumen open flow or access path between the first and second body lumens 150, 160, as discussed above. Various medical advantages may be realized by including multiple openings within the cylindrical saddle region 120. For example, the multiple openings may allow non-competing (e.g., bi-directional) flow and/or circulation of fluids between the first and second body lumens 150, 160. The position of the third, fourth and fifth openings 522, 524, 526 at various radial and longitudinal locations along the cylindrical saddle region 520 may allow gravitational forces to maintain an open flow path between the first and second body lumens 150, 160 regardless of the position (e.g., laying down, sitting, standing, etc.) of the patient. Any of the third, fourth and/or fifth openings 522, 524, 526 may provide separate access paths between the first and second body lumens 150, 160 for the simultaneous introduction and manipulation of two or more medical tools. Such tools may be selectively introduced through the various openings depending on the portion (e.g., upper, lower, side) of the second body lumen to be accessed. In one embodiment, the third, fourth and fifth openings 522, 524, 526 may provide improved drainage of large and/or non-parallel body lumens by maintaining at least one of the openings oriented along the outer radius of the cylindrical saddle region 520, thereby maximizing a diameter of the opening. The third, fourth and fifth openings 522, 524, 526 may also allow drainage from separate regions of a body lumen, including, for example, drainage of multiple cysts within a body lumen without introducing multiple medical devices and/or performing multiple medical procedures.

It should be appreciated that the present disclosure is not limited to including a plurality of openings (e.g., third, fourth and fifth openings, etc.) at various locations along a length of the cylindrical saddle region of the medical device 500 depicted in FIGS. 7A-7B, but may be included within any of the medical devices 100, 200, 300, 400, as discussed above.

In various embodiments, a medical device (e.g., medical devices 100, 200, 300, 400 or 500) of the present disclosure may include a first retention member configured to contact an inner surface of a second body lumen at a first location and a second retention member configured to contact an inner surface of the second body lumen at a second location such that the cylindrical saddle region and third opening extend into a first body lumen. Referring to FIG. 8A, in use and by way of example medical device 100, an endoscope 180 may be advanced through the esophagus into a first body lumen 150 (e.g., stomach) to image the tissue walls 152, 162 of the first and second body lumens 150, 160, as discussed above. A tissue-penetrating element (e.g., needle, etc.; not shown) comprising a proximal end, a sharpened distal end and a lumen extending therebetween, may then be advanced through a working channel of the endoscope such that the sharpened distal end penetrates the first tissue wall 152 at a first location 154, penetrates the second tissue wall 162 at a first location 164 and extends into the second body lumen 160. The tissue-penetrating element may then be bent (e.g., torqued or retroflexed) and advanced within the second body lumen 160 such that the sharpened distal end penetrates the second tissue wall 162 at a second location 166 (e.g., different than the first location 164), penetrates the first tissue wall 152 at a second location 156 (e.g., different than the first location 154) and extends into the first body lumen 150. The tissue-penetrating element may then be bent within the first body lumen 150 and further advanced through the previously formed opening between the first and second tissue walls (e.g., the first location 154 of the first tissue wall 152, and the first location 164 of the second tissue wall 162) to extend into the second body lumen 160. A guidewire 184 may be advanced through the lumen of the tissue-penetrating element (not shown) to position a distal portion 186 of the guidewire 184 within the second body lumen 160. The tissue-penetrating element (not shown) may then be retracted over the guidewire 184, and a sheath (not shown) advanced over the guidewire 184 to position a distal end of the sheath within the first second body lumen 160. A delivery system (not shown) may then be advanced over the guidewire 184 and through the sheath to position a distal end of the delivery system within the second body lumen 160. The delivery system may include an inner member with a lumen extending therethrough to receive the guidewire 184, and an outer member disposed coaxially about the inner member. A medical device 100 may be loaded onto the delivery system in a first configuration between the inner and outer member. Optionally, the delivery system may be advanced without a sheath along the same path with use of the guidewire 184.

Referring to FIG. 8B, a first portion 112 of the medical device 100 may be exposed from within the delivery system such that the first portion 112 forms a first retention member 114 within the second body lumen 160 at the first location 164 of the second tissue wall 162. The delivery system and sheath may then be proximally retracted (e.g., pulled) to place the first retention member 114 in contact with an inner surface 168 of the second body lumen 160. The sheath and delivery system may be further proximally retracted into and through the first body lumen 150 to position their respective distal ends within the second body lumen 160 at the second location 166 of the second tissue wall 162. A second portion of the 116 of the of the medical device 100 may be exposed from within the delivery system to form a second retention member 118 within the second body lumen 160 at the second location 166 of the second tissue wall 162. With the first and second retention members 114, 118 disposed within their respective locations 164, 166 of the second body lumen 160, the medical device 100 may assume a curved or “U-shaped” configuration such that a portion of the cylindrical saddle region 120, including the third opening 122, is disposed within the first body lumen 150, thereby providing an open flow path or access path between the first and second body lumens 150, 160. As discussed above, the first opening 113, second opening 117 and third opening 122 of the cylindrical saddle region, may allow non-competing (e.g., bi-directional) flow and/or circulation of fluids between the first and second body lumens 150, 160. In an alternative embodiment, without a sheath, the delivery system for a medical device, may be retracted along the guidewire and along the same path, with the first and second retention members deployed, as described above, by retracting the outer member from about the inner member of the delivery system to deploy the medical device.

In addition to providing a bi-directional flow path, the open interior passage 130 of the medical device 100 may provide separate access paths between the first and second body lumens 150, 160 for the simultaneous introduction and manipulation of two or more medical tools. For example, a first medical tool (e.g., forceps, cutting element, etc.) may be advanced from the first body lumen 150 into the second body lumen 160 through the third opening 122 of the cylindrical saddle region 120 and first openings 113 (e.g., through a portion of the open interior passage 130), and a second medical tool (e.g., suction tube, lavage tube, etc.) may be advanced from the first body lumen 150 into the second body lumen 160 through the third opening of the cylindrical saddle region and second opening 117 (e.g., through another portion of the open interior passage).

Although FIGS. 8A-8B outline exemplary steps by which a medical device 100 of the present disclosure may be deployed between first and second body lumens 150, 160, in various embodiments the same (or similar) steps may be used to deploy any of the medical devices 200, 300, 400, 500, as discussed above.

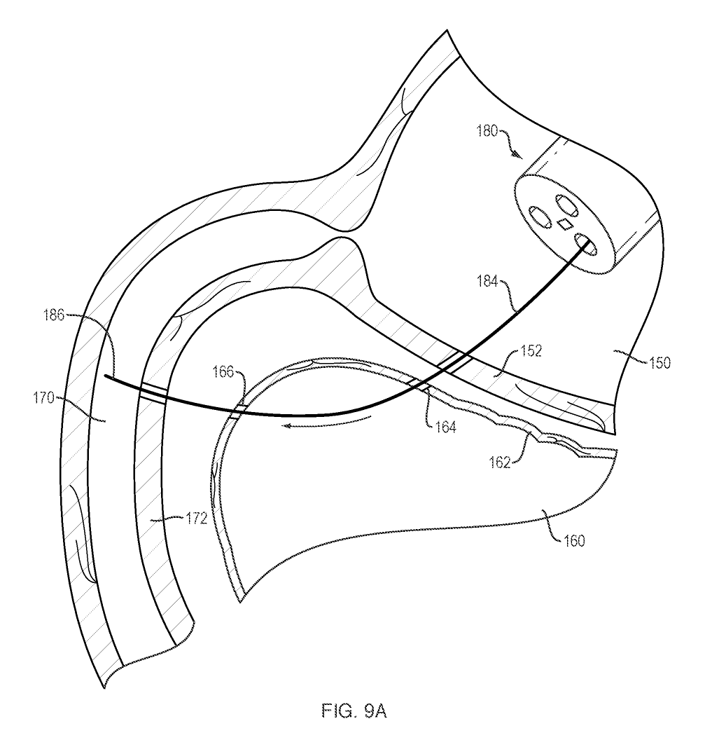

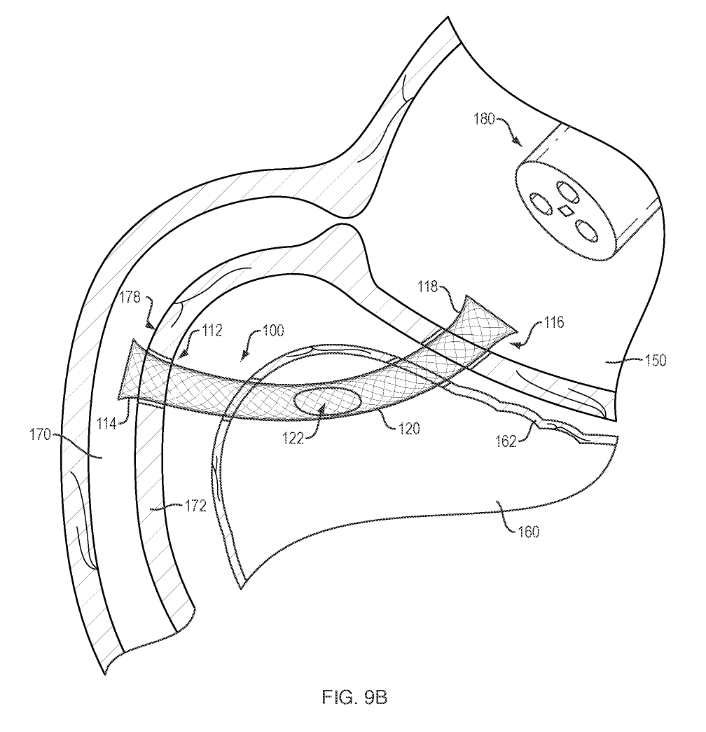

Referring to FIGS. 9A-9B, in one embodiment, a medical device 100 of the present disclosure may include a first retention member 114 configured to contact an inner surface of a first body lumen and a second retention member 118 configured to contact an inner surface of a third body such that the cylindrical saddle region 120 and third opening 122 may extend into and through a second body lumen. In use and by way of example, an endoscope 180 may be advanced through the esophagus into a first body lumen 150 (e.g., stomach) to image the tissue walls 152, 162, 172 of first, second and third body lumens 150, 160, 170, as discussed above. A tissue-penetrating element (e.g., needle, etc.) comprising a proximal end, a sharpened distal end and a lumen extending therebetween, may then be advanced through a working channel of the endoscope such that the sharpened distal end penetrates the tissue wall 152 of the first body lumen 150. The tissue-penetrating element may be further advanced such that the sharpened distal end penetrates the tissue wall 162 of the second body lumen 160 (e.g., pancreatic pseudocyst) at a first location 164, extends through a portion of the second body lumen 160 and penetrates the tissue wall 162 of the second body lumen 160 at a second location 166. The tissue-penetrating element may be further advanced such that that sharpened distal end penetrates the third tissue wall 172 and extends into the third body lumen 170 (e.g., duodenum). A guidewire 184 may be advanced through the lumen of the tissue-penetrating element (not shown) to position a distal portion 186 of the guidewire 184 within the third body lumen 170 (FIG. 9A). The tissue-penetrating element (not shown) may then be retracted over the guidewire 184, and a sheath advanced over the guidewire 184 to position a distal end of the sheath within the first second body lumen 160 (not shown). A delivery system (not shown) may then be advanced over the guidewire 184 and through the sheath to position a distal end of the delivery system within the third body lumen 170. The delivery system may include an inner member with a lumen extending therethrough to receive the guidewire 184, and an outer member disposed coaxially about the inner member. A medical device 100 may be loaded onto the delivery system in a first configuration between the inner and outer member. Optionally, the delivery system may be advanced without a sheath along the same path over guidewire 184.

Referring to FIG. 9B, a first portion 112 of the medical device 100 may be exposed from within the delivery system such that the first portion 112 forms a first retention member 114 within the third body lumen 170. The delivery system and sheath may then be proximally retracted (e.g., pulled) to place the first retention member 114 in contact with an inner surface 178 of the third body lumen 170. The sheath and delivery system may be further proximally retracted into and through the second body lumen 160 to position their respective distal ends within the first body lumen 150. A second portion of the 116 of the of the medical device 100 may be exposed from within the delivery system to form a second retention member 118 within the first body lumen 150. With the first and second retention members 114, 118 disposed within the third and first body lumens 170, 150, respectively, the cylindrical saddle region 120, including the third opening 122, may be disposed within the second body lumen 160, thereby providing an open flow path or access path between the first, second and third body lumens 150, 160, 170. In an alternative embodiment, without a sheath, the delivery system for a medical device, may be retracted along the guidewire and along the same path, with the first and second retention members deployed, as described above, by retracting the outer member from about the inner member of the delivery system to deploy the medical device.

Various medical advantages may be realized by establishing an open flow path or access path between three body lumens with a medical device 100 of the present disclosure. For example, the multiple openings may allow non-competing efferent (e.g., from the first body lumen 150, into the second body lumen 160) and afferent (e.g., from the second body lumen 160, into the third body lumen 170) flow. In addition to facilitating efficient removal or drainage of the contents of the second body lumen 160 into the third body lumen 170, a continued flow and circulation of high pH stomach fluids of the body lumen 150 (e.g., stomach) within and through the second body lumen 160 may neutralize or sanitize the potentially septic contents of the second body lumen. In addition, or alternatively, the circulating fluids from the first body lumen may increase the viscosity (e.g., degrade large pieces of debris, etc.) of the contents of the second body lumen, thereby further facilitating afferent flow into the third body lumen 170.

Although FIGS. 9A-9B outline exemplary steps by which a medical device 100 of the present disclosure may be deployed between first and second body lumens 150, 160, in various embodiments the same (or similar) steps may be used to deploy any of the medical devices 200, 300, 400, as discussed above.

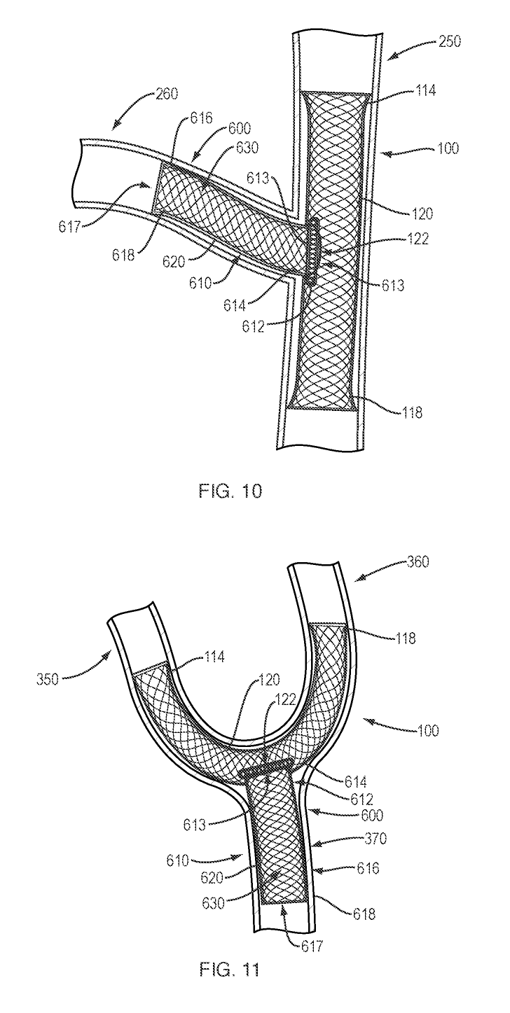

Referring to FIG. 10, in one embodiment, a medical device 100 of the present disclosure may include a first retention member 114 configured to contact an inner circumference of a first portion of a first body lumen or vessel 250 (e.g., the common bile duct), and a second retention member 118 configured to contact an inner circumference of a second portion of the first body lumen 250 such that the third opening 122 of the cylindrical saddle region 120 may at least partially overlap a junction of the first body lumen with a second body lumen 260 (e.g., the cystic duct). The third opening 122 of the cylindrical saddle region 120 may be configured to receive (e.g., connect to, attach to, etc.) a portion of a second medical device 600 to provide a combined open flow or access passage between the first and second body lumens 250, 260. For example, in one embodiment, the second medical device 600 may include an elongate tubular member 610 configured to move between a first (e.g., constrained, collapsed, non-expanded) configuration and a second (e.g., non-constrained, expanded) configuration. In the second configuration, a first (e.g., proximal) portion 612 of the elongate tubular member 610 may form a first (e.g., proximal) retention member 614 defining a first (e.g., proximal) opening 613, and a second (e.g., distal) portion 616 of the elongate tubular member 610 may form a second (e.g., distal) retention member 618 defining a second (e.g., distal) opening 617. A cylindrical saddle region 620 may extend between the first and second retention members 614, 618 to define an open interior passage 630 (e.g., channel, lumen, etc.) therebetween. The first retention member 614 may form a first flared end or single-wall flange structure, as discussed above, configured to engage an inner surface of the medical device 100 around a circumference of the third opening 122 of the cylindrical saddle region 120. The second retention member 618 may form a second flared end (e.g., flared flange structure) configured to atraumatically engage an inner circumference of the second body lumen. The approximately T-shaped configuration of the combined first and second medical devices 100, 600 may maintain an open flow or access passage through the first and second body lumens. As discussed above, the open flow passage may maintain the first and second body lumens in an open configuration to facilitate drainage therebetween, including, for example, the drainage of bile from the gallbladder into the common bile duct. In addition, or alternatively, the open access passage may allow one or more medical tools to be introduced into the gallbladder to remove obstructions, including, for example, gallstones.

Referring to FIG. 11, in one embodiment, a medical device 100 of the present disclosure may include a first retention member 114 configured to contact an inner circumference of a portion of a first body lumen 350 (e.g., the left hepatic duct), and a second retention member 118 configured to contact an inner circumference of a portion of a second body lumen 360 (e.g., the right hepatic duct) such that the third opening 122 of the cylindrical saddle region 120 at least partially overlaps a junction of the first and second body lumens 350, 360 with a third body lumen 370 (e.g., the common bile duct). The third opening 122 of the cylindrical saddle region 120 may be configured to receive (e.g., connect to, attach to, etc.) a portion of a second medical device 600 to provide a combined open flow or access passage between the first and second body lumens. For example, in one embodiment, the second medical device 600 may include an elongate tubular member 610 configured to move between a first (e.g., constrained, collapsed, non-expanded) configuration and a second (e.g., non-constrained, expanded) configuration. In the second configuration, a first (e.g., proximal) portion 612 of the elongate tubular member 610 may form a first (e.g., proximal) retention member 614 defining a first (e.g., proximal) opening 613, and a second (e.g., distal) portion 616 of the elongate tubular member 610 may form a second (e.g., distal) retention member 618 defining a second (e.g., distal) opening 617. A cylindrical saddle region 620 may extend between the first and second retention members 614, 618 to define an open interior passage 630 (e.g., channel, lumen, etc.) therebetween. The first retention member 614 may form a first flared end or single-wall flange structure configured to engage an inner surface of the medical device 100 around a circumference of the third opening 122 of the cylindrical saddle region 120, as discussed above. The second retention members 618 may form a second flared end (e.g., flared flange structure) configured to atraumatically engage an inner circumference of the third body lumen 350. The approximately Y-shaped configuration of the combined first and second medical devices 100, 600 may maintain an open flow or access passage through the first, second and third body lumens 350, 360, 370. As discussed above, the open flow passage may maintain the first, second and third body lumens in an open configuration to facilitate drainage therebetween, including, for example, the drainage of bile from the liver through into the common bile duct. Although the embodiments in FIGS. 10-11 depict medical devices to use with multiple body lumens or portions of multiple lumens, similar devices may be used with multiple portions or branches of the same body lumen.

Although the various opening within the cylindrical saddle region are depicted as substantially spherical, in various embodiments the one or more of the openings may include any number of sizes and/or shapes, including, but not limited to, triangles, squares, rectangles, pentagons, hexagons, octagons, ovals, oblong spheres, and combinations or variations thereof.

Although the medical devices 100, 200, 300, 400, 500, 600 disclosed herein are generally depicted as including woven, knitted or braided filaments (e.g., nitinol, etc.), in various embodiments, the medical devices may include laser cut designs which may or may not change in length (e.g., shorten) as the medical device moves from the first configuration to the second configuration. The medical devices in various configurations may be self-expanding or expandable such as balloon-expandable. A covering or coating may be disposed along an inner and/or outer wall of any of the medical devices to fully or partially enclose the weave of the medical devices. The covering or coating may comprise a variety of non-degradable and biocompatible polymeric materials (e.g., upon exposure to bodily fluids such as bile), including, for example, silicones, rubbers, polyethylenes and thermoplastic elastomers.

In various embodiments, the proximal and distal retention members may include various configurations, such that one or more of the single or double-wall flange structures extend radially at an angle that is not necessarily perpendicular to the elongate tubular body and/or include surfaces that are not necessarily planar. For example, either (or both) proximal and distal retention member (e.g., single or double-wall flange structures) may extend outward towards an end of the elongate body, back towards a center portion of the elongate tubular member, or change directions one or more times along the length of the flange structure (e.g., introducing a single or multiple inflection points and segment lengths along the flange structure where the flange changes direction), or in various combinations of each.

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A medical device, comprising:
an elongate tubular member having a first collapsed configuration;
the elongate tubular member having a second expanded configuration with a first portion of the elongate tubular member expanded into a first retention member defining a first opening, a second portion of the elongate tubular member expanded into a second retention member defining a second opening, and a cylindrical saddle region having a circumference and longitudinal axis extending between the first and second retention members;
wherein the cylindrical saddle region includes a third opening defined therein;
wherein the first retention member, second retention member and cylindrical saddle region are covered apart from the first, second and third openings; and
wherein the proximal retention member, distal retention member and cylindrical saddle region define an open interior passage therethrough.

2. The medical device of claim 1, wherein the first retention member includes a first flared flange structure, and the second retention members includes a second flared flange structure.

3. The medical device of claim 1, wherein the first retention member includes a first single-wall flange structure, and the second retention member includes a second single-wall flange structure.

4. The medical device of claim 1, wherein the first retention member includes a first double-wall flange structure, and the second retention member includes a second double-wall flange structure.

5. The medical device of claim 1, wherein the cylindrical saddle region includes a constant outer diameter.

6. The medical device of claim 1, wherein the cylindrical saddle region includes a varying outer diameter.

7. The medical device of claim 1, further comprising at least one valve disposed within the open interior passage of the elongate tubular member.

8. The medical device of claim 1, further comprising at least one valve disposed within at least one of the first, second or third openings.

9. The medical device of claim 1, wherein the third opening is positioned at an approximate midpoint of the circumference of the cylindrical saddle region.

10. The medical device of claim 1, wherein the cylindrical saddle region includes fourth and fifth openings positioned at different radial locations relative to the longitudinal axis of the cylindrical saddle region.

11. The medical device of claim 1, wherein at least a portion of the first retention member is configured to contact an inner surface of a first body lumen at a first location, at least a portion of the second retention member is configured to contact the inner surface of the first body lumen at a second location, and the third opening of the cylindrical saddle region is configured to open into a second body lumen.

12. The medical device of claim 1, wherein at least a portion of the first retention member is configured to contact an inner surface of a second distal body lumen at a first location, at least a portion of the second retention member is configured to contact the inner surface of the second body lumen at a second location, and the third opening of the cylindrical saddle region is configured to open into a first proximal body lumen.

13. The medical device of claim 1, wherein the first retention member is configured to contact an inner circumference of a first body lumen, the second retention member is configured to contact an inner circumference of a second body lumen, and the third opening of the cylindrical saddle region is configured to at least partially overlap a junction of the first and second body lumens with a third body lumen.

14. The medical device of claim 13, wherein the third opening of the cylindrical saddle region is configured to receive a portion of a second medical device disposed within the second body lumen.

15. The medical device of claim 1, wherein the first retention member is configured to contact an inner circumference of a first body lumen, the second retention member is configured to contact an inner circumference of a second body lumen, and the third opening of the cylindrical saddle region is configured to at least partially overlap a junction of the first and second body lumens with a third body lumen.

16. The medical device of claim 15, wherein the third opening of the cylindrical saddle region is configured to receive a portion of a second medical device within the third body lumen.

17. A method, comprising:
advancing a medical device into a first body lumen, wherein the medical device includes an elongate tubular member having a first collapsed configuration and a second expanded configuration;
advancing the medical device into a second body lumen through an opening in the first body lumen at a first location and an opening in the second body lumen at a first location;
further advancing the medical device from the second body lumen into the first body lumen through an opening in the second body lumen at a second location and an opening in the first body lumen at a second location;
expanding a distal portion of the elongate tubular member to the second configuration, such that a distal retention member of the member is deployed within the first body lumen at the second location; and
expanding a proximal portion of the elongate tubular member to the second configuration, such that a proximal retention member of the member is deployed within the first body lumen at the first location.

18. The method of claim 17, wherein the elongate tubular member includes a cylindrical saddle region extending between the proximal and distal retention members, openings at the distal and proximal portions open into the first body lumen, and wherein an opening of the cylindrical saddle region opens into the second body lumen when the proximal and distal retention members are disposed within the first body lumen.

19. A method, comprising:
advancing a medical device into a first body lumen, wherein the medical device includes an elongate tubular member having a first collapsed configuration and a second expanded configuration;
advancing the medical device into a second body lumen through an opening in the first body lumen at a first location and an opening in the second body lumen at a first location;
further advancing the medical device from the second body lumen into the first body lumen through an opening in the second body lumen at a second location and an opening in the first body lumen at a second location;
further advancing the medical device from the first body lumen into the second body lumen through the opening in the first body lumen at the first location and the opening in the second body lumen at the first location;
deploying a distal retention member of the medical device within the second body lumen at the first location; and
deploying a proximal retention member of the medical device within the second body lumen at the second location.

20. The method of claim 19, wherein the elongate tubular member includes a cylindrical saddle region extending between the proximal and distal retention members, openings at the distal and proximal portions open into the second body lumen, and wherein an opening of the cylindrical saddle region opens into the first body lumen when the proximal and distal retention members are disposed within the second body lumen.
